(19) United States
(12) Patent Application Publication
(10) Pub. No.: US 2018/0064353 A1
(43) Pub. Date: Mar. 8, 2018

(54) PHYSIOLOGICAL STATUS MONITORING SYSTEM

(71) Applicant: TINA SU, TAICHUNG CITY (TW)

(72) Inventor: TINA SU, TAICHUNG CITY (TW)

(21) Appl. No.: 15/802,685

(22) Filed: Nov. 3, 2017

Related U.S. Application Data
(63) Continuation-in-part of application No. 15/136,977, filed on Apr. 24, 2016.

(51) Int. Cl. A61B 5/024; A61B 5/044; G06F 19/00; A61B 5/0205

(52) U.S. Cl. CPC A61B 5/02438; A61B 5/044; A61B 5/1118; A61B 5/0205; G06F 19/3418

(57) ABSTRACT

A physiological status monitoring system includes a clothing and a mobile device. The clothing has a display unit connected to the outside thereof, and a sensor is located in the clothing. The sensor includes a control unit, an emitting unit and a power supply unit received therein. The control unit has a flashing program, a warning program, a safety program and a racing program written therein. The wearer holds the mobile device which is connected to the sensor wirelessly and has a receiving unit. The sensor detects physiological data of the wearer, and the physiological data of the wearer is displayed on the display unit. The physiological data of the wearer is also sent to the mobile device. The wearer can be acknowledged his/her physiological status.

### FIELDS OF THE INVENTION

The present invention is a Continuation-In-Part application of applicant's former patent application Ser. No. 15/136,977, filed on Apr. 24, 2016, currently pending.

### BACKGROUND OF THE INVENTION

### Descriptions of Related Art

U.S. Patent Publication No. 2017/0199797 discloses “an ambulatory medical device” and comprises a plurality of subsystems comprising: at least one sensor configured to acquire data descriptive of a patient; a user interface; and at least one processor coupled to the at least one sensor and the user interface and configured to identify subsystem status information descriptive of an operational status of each subsystem of the plurality of subsystems and to provide a device health report for the ambulatory medical device via the user interface, the device health report being based on the operational status of each subsystem.

The present invention intends to provide a physiological status monitoring system which provides information to the user and other people so as to properly take care of the user in time.

### SUMMARY OF THE INVENTION

The present invention relates to a physiological status monitoring system, and comprises a clothing and a mobile device. The clothing has a display unit connected to the outside thereof, and a sensor is located in the clothing and wirelessly connected with the display unit. The sensor has a control unit, an emitting unit and a power supply unit received therein. The sensor is connected to the display unit via the control unit. The power supply unit provides electric power to the display unit. The control unit has a flashing program, a warning program, a safety program and a racing program written therein. The mobile device is connected to the sensor wirelessly and has a receiving unit received therein. The receiving unit and the emitting unit emit data wirelessly. A wearer holds the mobile device, and the sensor detects physiological data of the wearer. The physiological data of the wearer is displayed on the display unit and sent to the mobile device.

Alternatively, the present invention provides a physiological status monitoring system, and comprises a clothing and a mobile device. The clothing has a display unit connected to the outside thereof, and a sensor is located in the clothing. The sensor has a control unit, an emitting unit and a power supply unit received therein. The sensor is connected to the display unit via the control unit. The power supply unit provides electric power to the display unit. The display unit and the sensor are connected to each other by a cable which is located within the clothing. The mobile device is connected to the sensor wirelessly and has a receiving unit received therein. The receiving unit and the emitting unit emit data wirelessly. A wearer holds the mobile device, and the sensor detects physiological data of the wearer. The physiological data of the wearer is displayed on the display unit and sent to the mobile device.

The primary object of the present invention is to provide a physiological status monitoring system, wherein the physiological status of the wearer can be detected by the sensor of the clothing that the wearer wears. The physiological status is sent to the display unit on the clothing and the mobile device so that the wearer is acknowledged his/her physiological status.

The present invention will become more apparent from the following description when taken in connection with the accompanying drawings which show, for purposes of illustration only, a preferred embodiment in accordance with the present invention.

### BRIEF DESCRIPTION OF THE DRAWINGS

FIG. 1 shows the components of the clothing of the physiological status monitoring system of the present invention;

FIG. 2 shows that the display unit is a FlexUPD;

FIG. 3 shows that the display unit is buttons of the clothing;

FIG. 4 shows that the display unit is light strips on the clothing;

FIG. 5 shows the cooperation between the clothing and the mobile device of the physiological status monitoring system of the present invention;

FIG. 6 shows that the wearer uses the physiological status monitoring system of the present invention when joining a racing competition;

FIG. 7 shows that the wearer uses the physiological status monitoring system of the present invention wherein the clothing is an arm warp;

FIG. 8 shows that the wearer uses the physiological status monitoring system of the present invention wherein the clothing is a sport wear;

FIG. 9 shows another angle of view of the clothing in FIG. 8;

FIG. 10 shows the components of the clothing of the second embodiment of the physiological status monitoring system of the present invention;

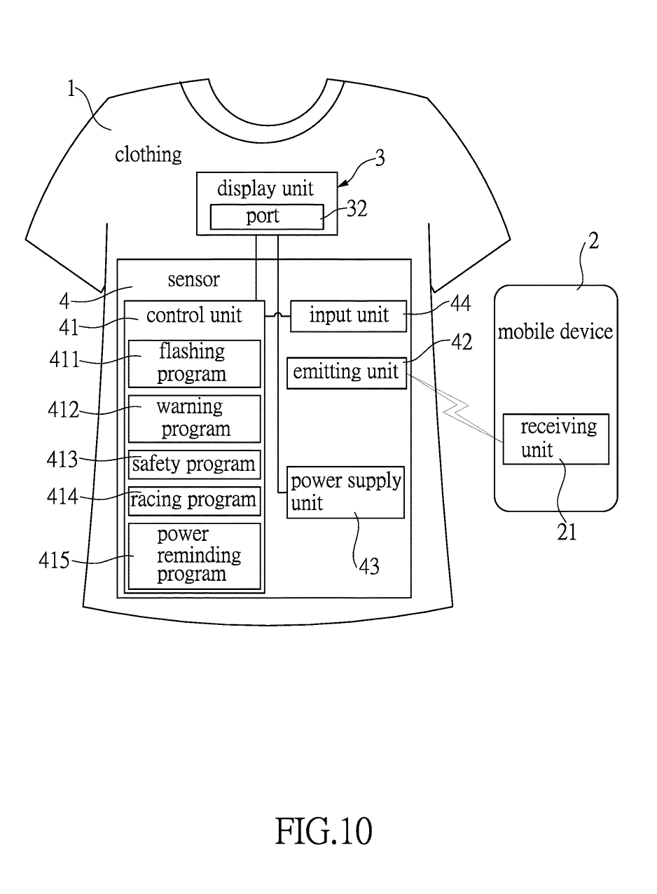

FIG. 11 shows that the display unit flashes when in lower power;

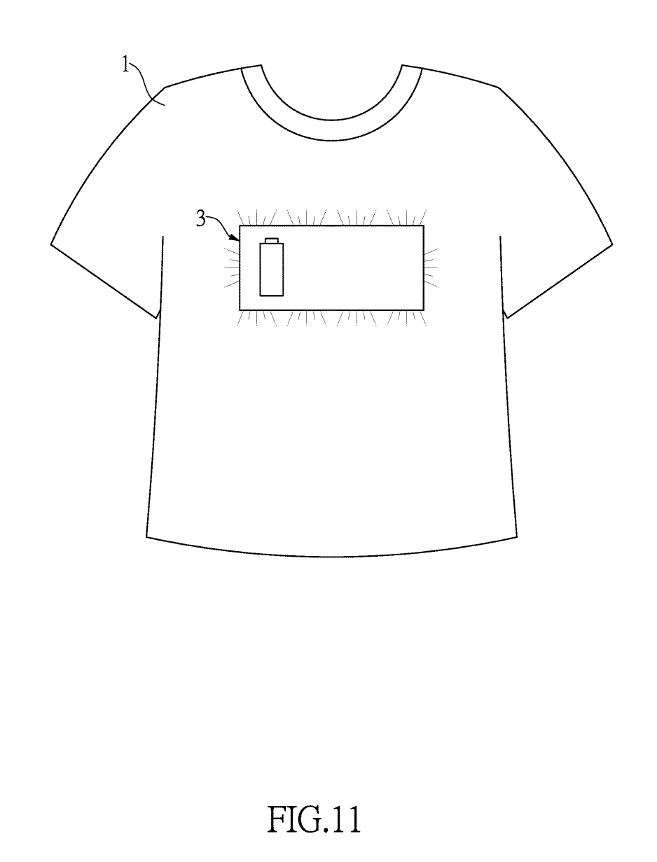

FIG. 12 shows that the cable is hidden in the clothing of the clothing of the physiological status monitoring system of the present invention;

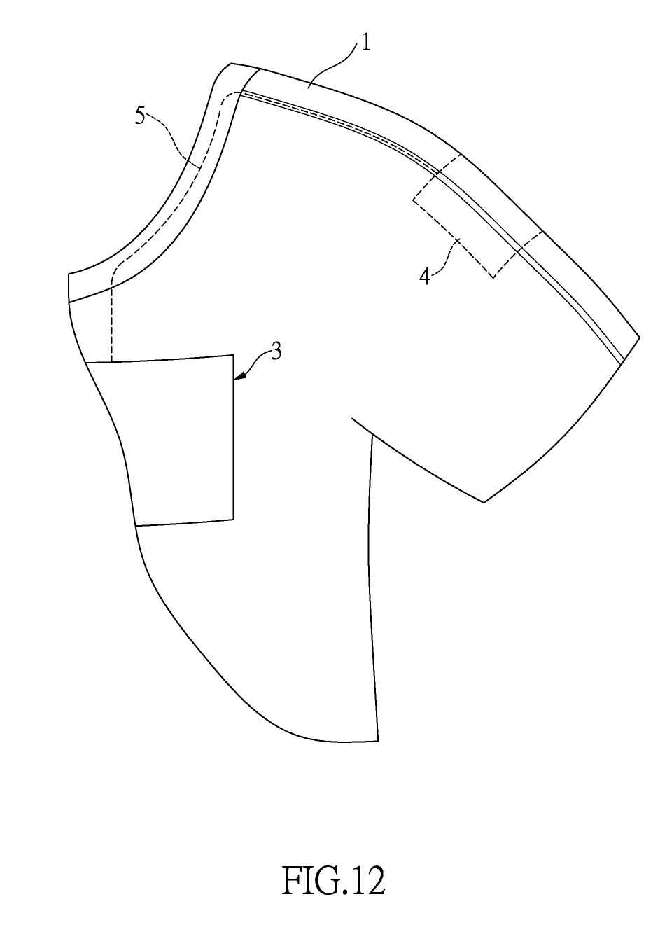

FIG. 13 shows a SIM card is to be inserted into the port of the display unit which is a FlexUPD;

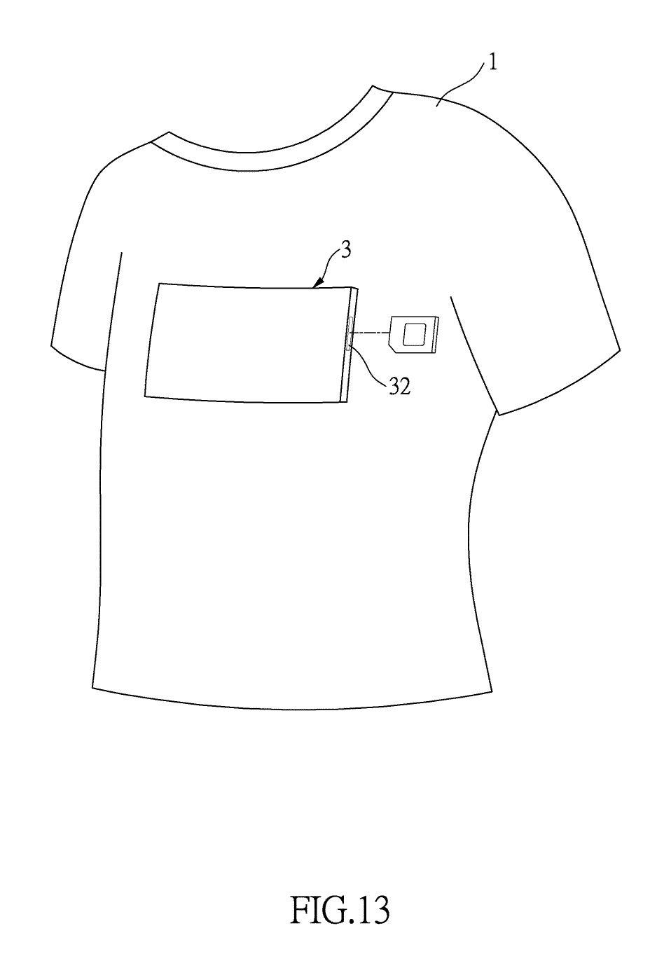

FIG. 14 shows a SIM card is to be inserted into the port of the display unit which is a button, and

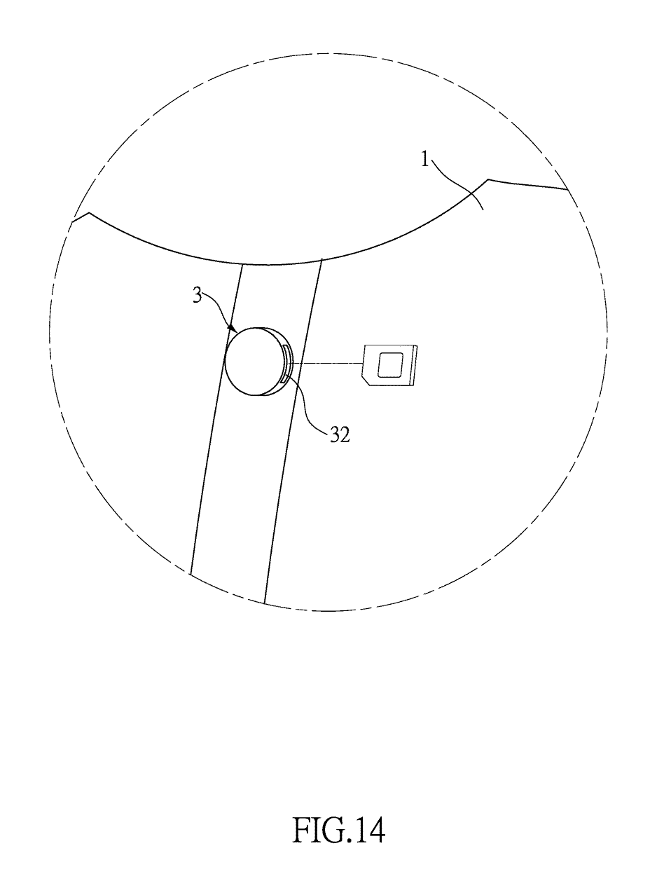

FIG. 15 shows a SIM card is to be inserted into the port of the display unit which includes multiple light strips.

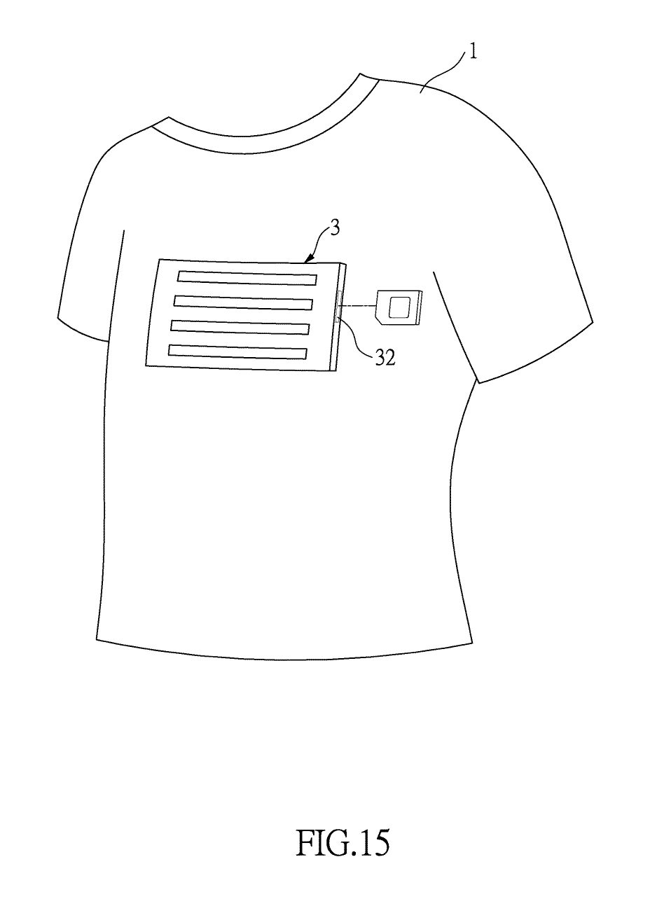

### DETAILED DESCRIPTION OF THE PREFERRED EMBODIMENT

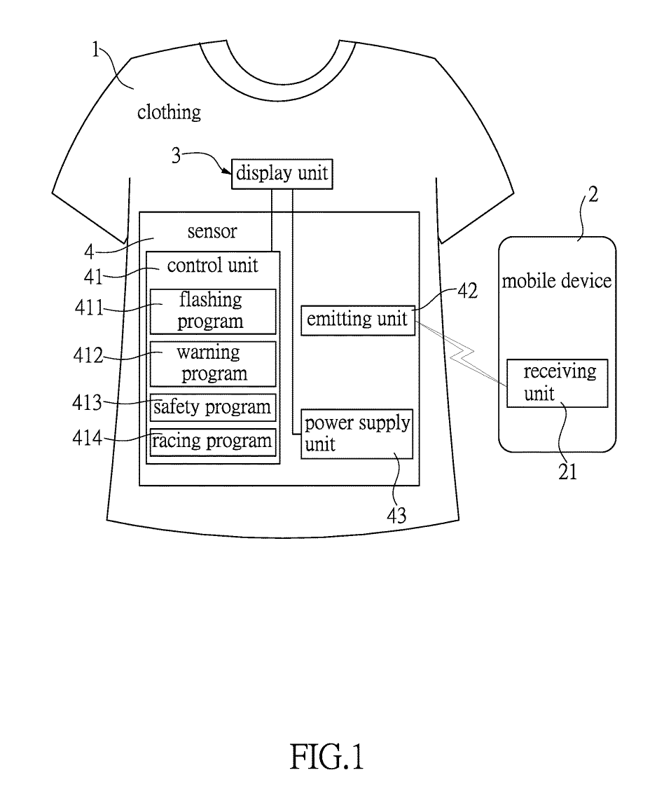

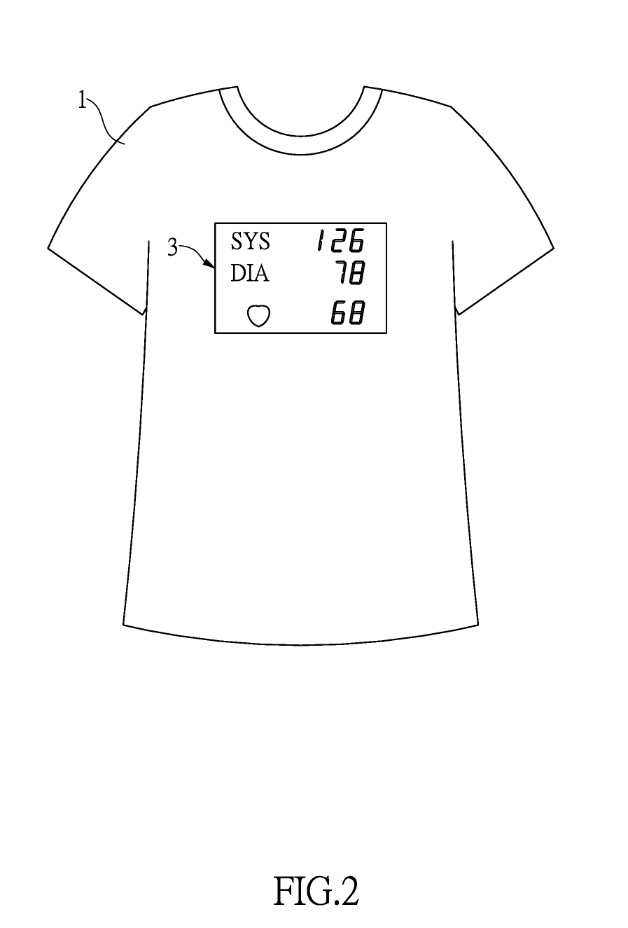

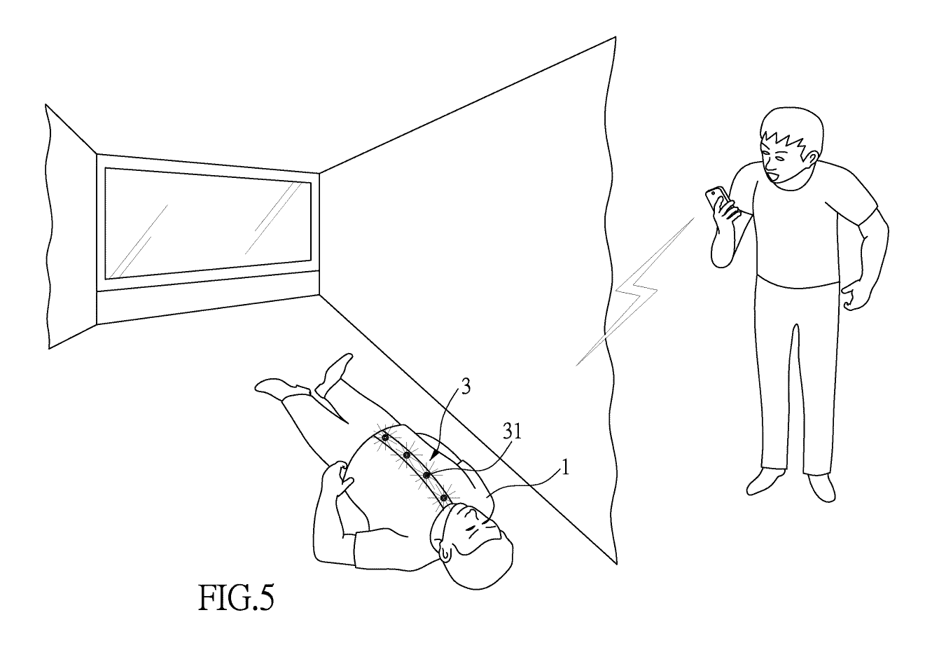

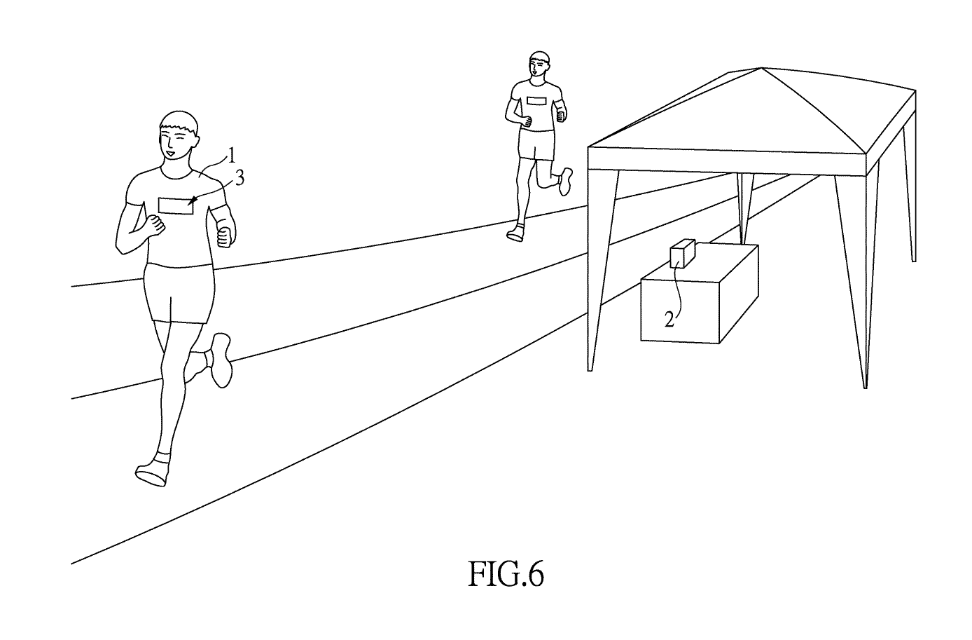

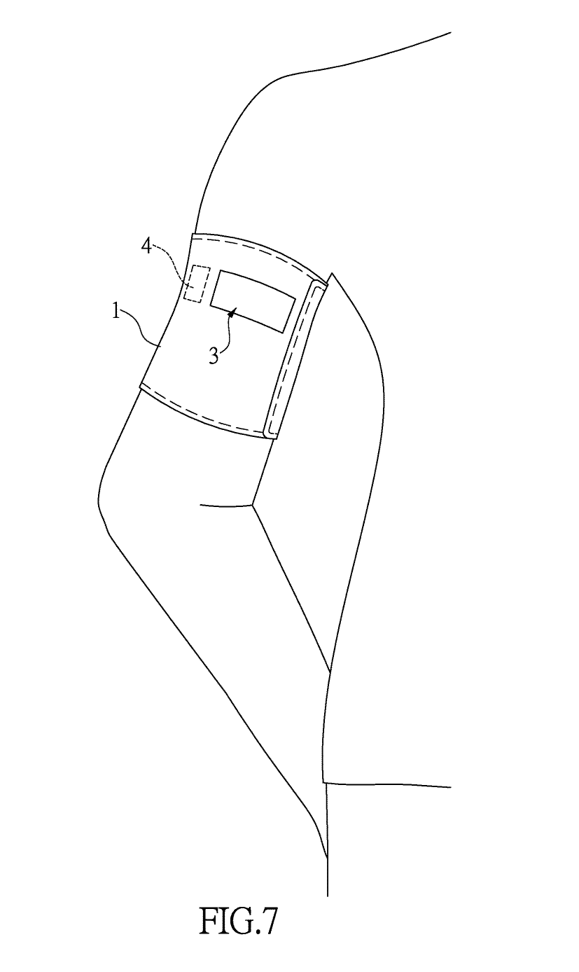

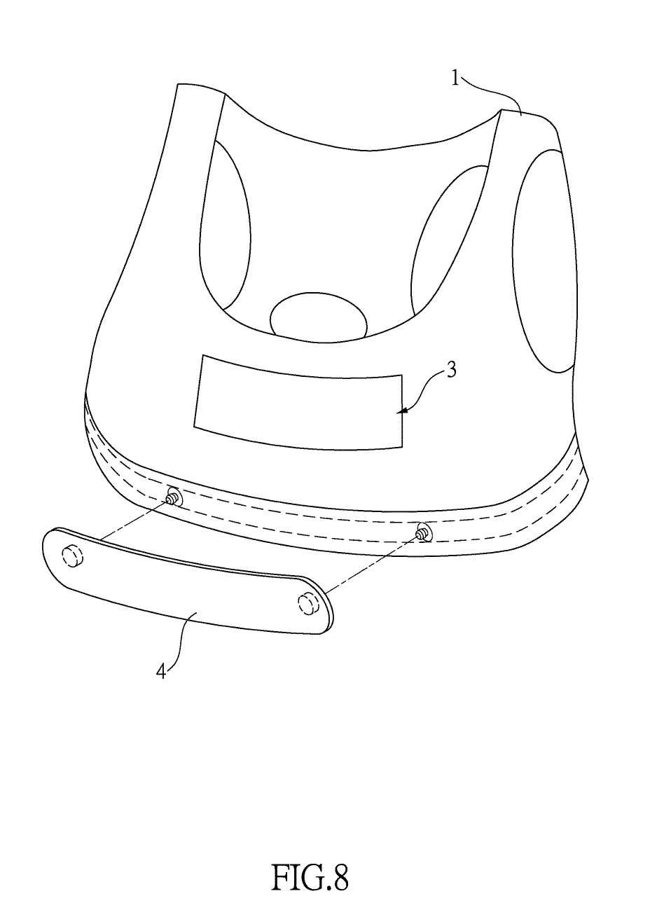

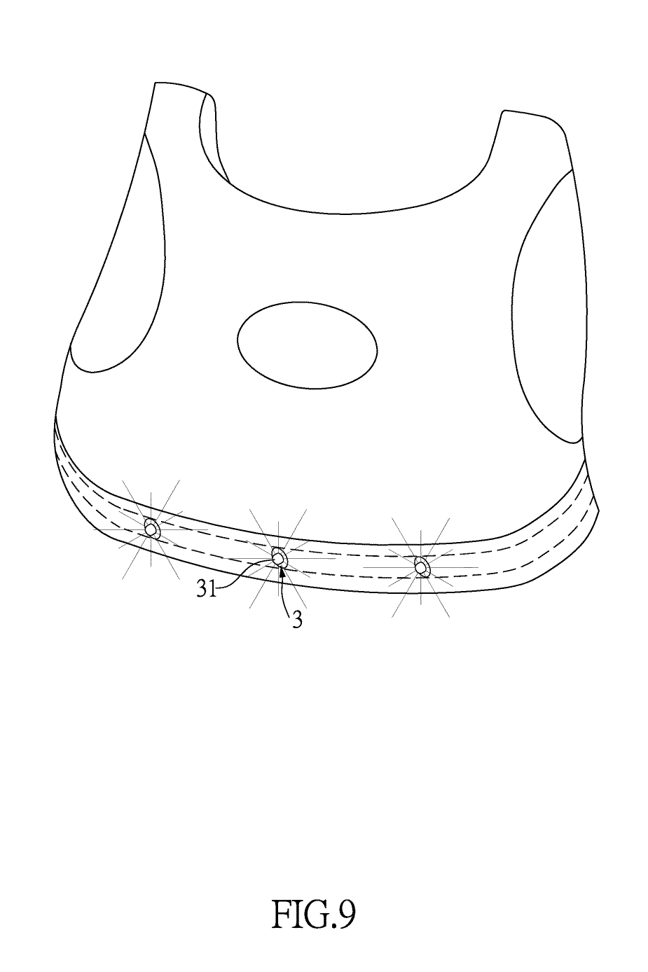

Referring to FIGS. 1 to 9, the physiological status monitoring system of the present invention comprises a clothing 1 and a mobile device 2. The clothing 1 has a display unit 3 connected to the outside thereof, and a sensor 4 is located in the clothing 1 and wirelessly connected with the display unit 3. The sensor 4 detects physiological data of the wearer wearing the clothing 1. The sensor 4 has a control unit 41, an emitting unit 42 and a power supply unit 43 received therein. The sensor 4 is connected to the display unit 3 via the control unit 41. The power supply unit 43 provides electric power to the display unit 3. The control unit 41 has a flashing program 411, a warning program 412, a safety program 413 and a racing program 414 written therein.

The mobile device 2 is connected to the sensor 4 wirelessly and has a receiving unit 21 received therein. The receiving unit 21 and the emitting unit 42 emit data wirelessly. The wearer holds the mobile device 2, and the sensor 4 detects physiological data of the wearer. The physiological data of the wearer is displayed on the display unit 3 and sent to the mobile device 2. Therefore, the wearer is acknowledged his/her physiological status. The mobile device 2 can also be held by another person so as to monitor the safety of the wearer.

The display unit 3 can be a FlexUPD a button or a light strip. The FlexUPD displays photos, characters and colors with changeable brightness. In application, the display unit 3 displays heart rate, body temperature, adrenaline, and calories of the wearer. The display unit 3 is able to display quick change of colors and brightness by the flashing program 411 to imply that the wearer does not wear the clothing 1 properly. The colors are red, yellow and green, wherein the red color means danger, the yellow color means warning, and the green color means safe. The display unit 3 displays changed colors by the warning program 412 to imply that the wearer has abnormal physiological status. In other words, the color changes from green to yellow and/or red. The display unit 3 maintains the colors unchanged by the safety program 413 to imply that the wearer has normal physiological status, such as the color maintains green. Therefore, other persons know the situation of the wearer. When multiple wearers participate a speed racing game, the display unit 3 displays loops/kilometers that each wearer finishes, and the judge can set the same color for the wearers who complete the same loops by the racing program 414. The audience can easily tell which wearers lead.

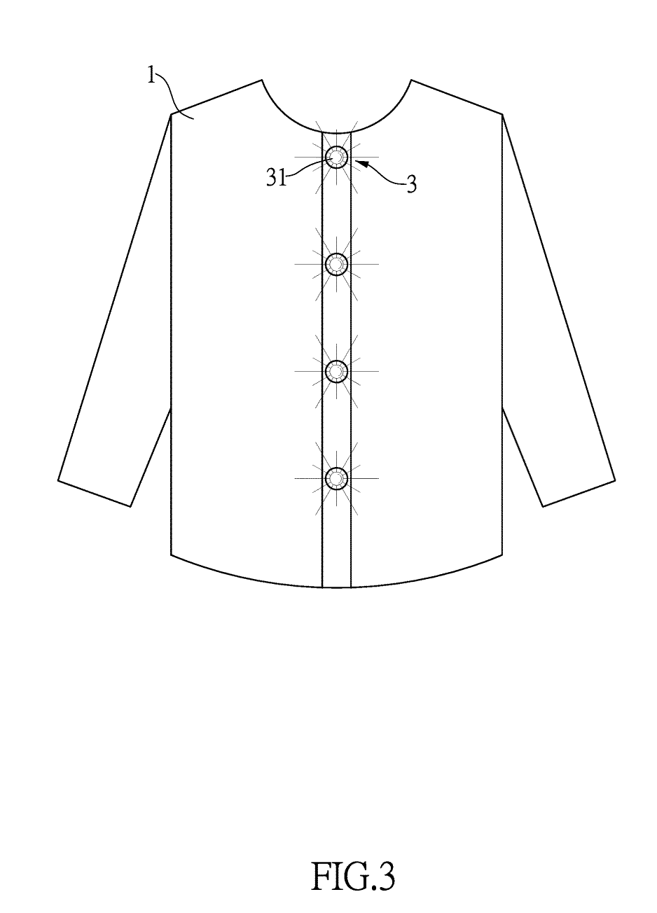

As mentioned before, the display unit 3 can be one of the buttons of the clothing 1 as shown in FIGS. 1 and 3, and includes at least one light emitting member 31 which emits light in colors and brightness. The buttons are located at the center of the clothing 1 and may include at least one Light Emitting Diode.

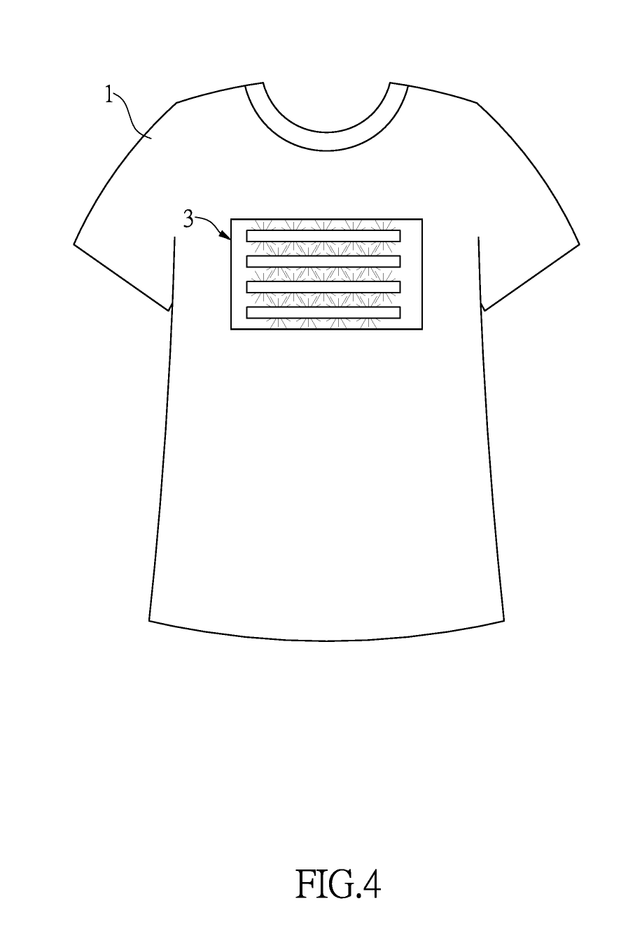

As mentioned before, the display unit 3 can be a light strip as shown in FIGS. 1 and 4, and emits light in colors and brightness. In one embodiment as shown in FIG. 4, there are four light strips in the display unit 3. The physiological data can be displayed by any of the FlexUPD, buttons or light strips so that the status of the wearer can be seen by others, and necessary help can be provided in time to the wearers.

The mobile device 2 can be a smart phone, a tablet, or an emitter. The mobile device 2 is electrically connected to the sensor 4 by way of Bluetooth, infrared ray, radio frequency or by connecting to a remote server. The sensor 4 sends data to the remote server and the mobile device 2 downwards the data from the remote server. Besides, the clothing 1 can be a cloth, a belt, a wrist band, an arm warp or a sport wear as shown in FIGS. 1, 5 to 9.

Alternatively, the present invention provides a second embodiment of the physiological status monitoring system, and comprises a clothing 1 and a mobile device 2. The clothing 1 has a display unit 3 connected to the outside thereof, and a sensor 4 is located in the clothing 1. The sensor 4 has a control unit 41, an emitting unit 42 and a power supply unit 43 received therein. The sensor 4 is connected to the display unit 3 via the control unit 41. The power supply unit 43 provides electric power to the display unit 3. The difference from the first embodiment is that the display unit 3 and the sensor 4 are connected to each other by a cable 5 which is located within or hidden in the clothing 1. The cable 5 is flexible and can be bent along with the wearer moving his/her body. The mobile device 2 is connected to the sensor 4 wirelessly and has a receiving unit 21 received therein. The receiving unit 21 and the emitting unit 42 emit data wirelessly. A wearer holds the mobile device 2, and the sensor 4 detects physiological data of the wearer. The physiological data of the wearer is displayed on the display unit 3 and sent to the mobile device 2.

The display unit 3 in this embodiment includes a port 32 which can be connected with an external hard drive, a SIM card or a USB. The sensor 4 has an input unit 44 which is connected to the control unit 41. The external hard drive, the SIM card or the USB reads data via the port 32 and sends the data to the input unit 44 of the sensor 4. The input unit 44 transfers the data to the control unit 41 and displays the data on the display unit 3. Again, the control unit 41 has a flashing program 411, a warning program 412, a safety program 413 and a racing program 414 written therein. The display unit 3 is a FlexUPD, a button or a light strip for displaying photos, characters and colors with changeable brightness. The features and functions of the display unit 3 are the same as that of the first embodiment. The mobile device 2 is a smart phone, a tablet, or an emitter, and the mobile device 2 is electrically connected to the sensor 4 by way of Bluetooth, infrared ray, radio frequency or by connecting to a remote server. The sensor 4 sends data to the remote server and the mobile device 2 downwards the data from the remote server. The clothing 1 can be a cloth, a belt, a wrist band, an arm warp or a sport wear.

The control unit 41 further includes a power reminding program 415 which provides information to be displayed on the display unit 3 when the power is low.

While we have shown and described the embodiment in accordance with the present invention, it should be clear to those skilled in the art that further embodiments may be made without departing from the scope of the present invention.

What is claimed is:

1. A physiological status monitoring system, comprising:
a clothing having a display unit connected to an outside thereof, a sensor located in the clothing and wirelessly connected with the display unit, the sensor having a control unit, an emitting unit and a power supply unit received therein, the sensor connected to the display unit via the control unit, the power supply unit providing electric power to the display unit, the control unit having a flashing program, a warning program, a safety program and a racing program written therein, and
a mobile device connected to the sensor wirelessly and having a receiving unit received therein, the receiving unit and the emitting unit emitting data wirelessly, wherein a wearer holds the mobile device, the sensor is adapted to detect physiological data of the wearer, the physiological data of the wearer is displayed on the display unit and sent to the mobile device.

2. The physiological status monitoring system as claimed in claim 1, wherein the display unit is a FlexUPD which displays photos, characters and colors with changeable brightness, the display unit displays heart rate, body temperature, adrenaline, and calories of the wearer, the display unit displays quick change of colors and brightness by the flashing program to imply that the wearer does not wear the clothing properly, the display unit displays changed colors by the warning program to imply that the wearer has abnormal physiological status, the display unit maintains the colors unchanged by the safety program to imply that the wearer has normal physiological status, the display unit displays loops/kilometers that the wearer finishes by the racing program.

3. The physiological status monitoring system as claimed in claim 1, wherein the display unit is one of buttons of the clothing and includes at least one light emitting member which emits light in colors and brightness.

4. The physiological status monitoring system as claimed in claim 1, wherein the display unit is a light strip which emits light in colors and brightness.

5. The physiological status monitoring system as claimed in claim 1, wherein the mobile device is a smart phone, a tablet, or an emitter, the mobile device is electrically connected to the sensor by way of Bluetooth, infrared ray, radio frequency or by connecting to a remote server, the sensor sends data to the remote server and the mobile device downwards the data from the remote server.

6. The physiological status monitoring system as claimed in claim 5, wherein the clothing is a cloth, a belt, a wrist band, an arm warp or a sport wear.

7. A physiological status monitoring system, comprising:
a clothing having a display unit connected to an outside thereof, a sensor located in the clothing, the sensor having a control unit, an emitting unit and a power supply unit received therein, the sensor connected to the display unit via the control unit, the power supply unit providing electric power to the display unit, the display unit and the sensor being connected to each other by a cable which is located within the clothing, and
a mobile device connected to the sensor wirelessly and having a receiving unit received therein, the receiving unit and the emitting unit emitting data wirelessly, wherein a wearer holds the mobile device, the sensor is adapted to detect physiological data of the wearer, the physiological data of the wearer is displayed on the display unit and sent to the mobile device.

8. The physiological status monitoring system as claimed in claim 7, wherein the display unit includes a port which is connected with an external hard drive, a SIM card or a USB, the sensor has an input unit which is connected to the control unit, the external hard drive, the SIM card or the USB reads data via the port and sends the data to the input unit of the sensor, the input unit transfers the data to the control unit and displays the data on the display unit, the control unit has a flashing program, a warning program, a safety program and a racing program written therein, the display unit is a FlexUPD which displays photos, characters and colors with changeable brightness, the display unit displays heart rate, body temperature, adrenaline, and calories of the wearer, the display unit displays quick change of colors and brightness by the flashing program to imply that the wearer does not wear the clothing properly, the display unit displays changed colors by the warning program to imply that the wearer has abnormal physiological status, the display unit maintains the colors unchanged by the safety program to imply that the wearer has normal physiological status, the display unit displays loops/kilometers that the wearer finishes by the racing program.

9. The physiological status monitoring system as claimed in claim 7, wherein the display unit includes a port which is connected with an external hard drive, a SIM card or a USB, the sensor has an input unit which is connected to the control unit, the external hard drive, the SIM card or the USB reads data via the port and sends the data to the input unit of the sensor, the input unit transfers the data to the control unit and displays the data on the display unit, the control unit has a flashing program, a warning program, a safety program and a racing program written therein, the display unit is one of buttons of the clothing and includes at least one light emitting member which emits light in colors and brightness.

10. The physiological status monitoring system as claimed in claim 7, wherein the display unit includes a port which is connected with an external hard drive, a SIM card or a USB, the sensor has an input unit which is connected to the control unit, the external hard drive, the SIM card or the USB reads data via the port and sends the data to the input unit of the sensor, the input unit transfers the data to the control unit and displays the data on the display unit, the control unit has a flashing program, a warning program, a safety program and a racing program written therein, the display unit is a light strip which emits light in colors and brightness.

11. The physiological status monitoring system as claimed in claim 7, wherein the mobile device is a smart phone, a tablet, or an emitter, the mobile device is electrically connected to the sensor by way of Bluetooth, infrared ray, radio frequency or by connecting to a remote server, the sensor sends data to the remote server and the mobile device downwards the data from the remote server.

12. The physiological status monitoring system as claimed in claim 11, wherein the clothing is a cloth, a belt, a wrist band, an arm warp or a sport wear.
